(19) United States
(12) Patent Application Publication
(10) Pub. No.: US 2025/0085227 A1
(43) Pub. Date: Mar. 13, 2025

(54) RAMAN SPECTRUM CLASSIFICATION METHOD, SPECIES BLOOD AND SEMEN CLASSIFICATION METHOD AND SPECIES CLASSIFICATION METHOD

(71) Applicant: Shanghai Maritime University, Shanghai (CN)

(72) Inventors: Rigui Zhou, Shanghai (CN); Pengju Ren, Shanghai (CN); Hanxuan Zhou, Shanghai (CN)

(73) Assignee: Shanghai Maritime University, Shanghai (CN)

(21) Appl. No.: 18/666,977

(22) Filed: May 17, 2024

(30) Foreign Application Priority Data
Sep. 7, 2023 (CN) 202311151223.3

(51) Int. Cl. G01N 21/65; G01N 33/49

(52) U.S. Cl. CPC G01N 21/65; G01N 33/49; G01N 2201/126

(57) ABSTRACT

The invention discloses a Raman spectrum classification method, a species blood and semen classification method and a species classification method. The Raman spectrum classification method comprises: first, acquiring Raman spectrum data as a training set and a test set; second, acquiring Raman spectrum data with obvious spectral peak information; then, inputting the spectrum data with the spectral peak information into a one-dimensional convolution and multi-head self-attention mechanism combined neural network model to train a classification model; and finally, inputting the Raman spectrum test set data to the trained classification model to obtain a classification result. The local peak feature information can be obtained by performing a convolution calculation on a spectrum, global peak correlation information can be obtained by performing a multi-head self-attention calculation, and the effect of multi-scale feature fusion is achieved, so that the local feature peak and the global peak correlation of a Raman spectrum can be combined.

### TECHNICAL FIELD

The present invention relates to the field of spectrum analysis, in particular to a Raman spectrum classification method, a species blood and semen classification method and a species classification method.

### BACKGROUND

A Raman spectrum is generated by performing scattering after a light source irradiates a substance molecule, and is widely used for analyzing structural information of a substance. According to differences in chemical structures and functional groups inside the irradiated substance, spectrum data generated thereby also has differences, and is also referred to as a “chemical fingerprint” of the substance. The Raman spectrum can not only perform qualitative analysis on a substance but also achieve quantitative analysis of the substance, and the content of the substance can be determined by the peak intensity of the spectrum. The Raman spectrum has advantages such as high efficiency, high sensitivity, being easy to perform sampling and no need to contact a sample, so that for some samples with infectivity, test personnel can be effectively protected, without damaging the samples. Therefore, the Raman spectrum is applied in fields such as food safety, medical detection and petrochemical industries.

The Raman spectrum is applied in the study of species blood identification. A vibration mode is generated by the interaction between laser photons and molecules in a blood sample, so as to generate Raman scattering. These pieces of scattering information provide vibration information such as hemoglobin, protein, lipid and sugar molecules in the blood, so that the concentration composition of the molecules in the blood can be determined, so as to analyze and determine the species to which the blood belongs. With the development of machine learning, the Raman spectrum can be used to obtain better identification results in a machine learning algorithm, in which a partial least squares regression analysis (PLS-DA) and principal component analysis (PCA) method are widely applied in blood spectrum analysis. However, the classic machine learning method has a good identification effect for small data samples, but also has certain limitations in large data samples.

As deep learning continues to advance, convolutional neural networks achieve superior effects in Raman spectrum classification tasks compared to classical machine learning methods. However, there is still a disadvantage of convolutional neural networks in Raman spectrum classification, the convolutional neural networks can only extract local peak feature information of a spectrum, and cannot capture a relationship between local and global feature information, and do not achieve the best classification effect.

### SUMMARY

The technical problem to be solved by the present invention is to provide a Raman spectrum classification method, which solves the problem in the prior art that a convolutional nerve cannot capture the relationship between local and global feature information of a spectrum.

The present invention adopts the following technical solutions to solve the described technical problem.

A Raman spectrum classification method, comprising:

first, acquiring several pieces of Raman spectrum data as a training set and a test set; second, after performing a series of preprocessing on the Raman spectrum data according to the existing spectrum quality, acquiring Raman spectrum data with obvious spectral peak information; then, inputting the Raman spectrum data with the obvious spectral peak information into a one-dimensional convolution and multi-head self-attention mechanism combined neural network model to train a classification model; and finally, inputting the Raman spectrum test set data to the trained classification model to obtain a final classification result.

The specific steps of performing preprocessing the spectrum data are as follows:

step 1, determining whether there is a baseline drift phenomenon in the Raman spectrum data, and if there is a baseline drift phenomenon, performing baseline correction processing;

step 2, determining whether there is an instrument noise in the Raman spectrum data acquisition process, and if there is a noise, performing noise removal processing on the Raman spectrum data;

step 3, replacing a spectrum dot with a spectrum intensity of a negative value with 0;

and step 4, normalizing the Raman spectrum obtained in step 3, and acquiring the Raman spectrum data X with obvious spectral peak information.

The specific steps of training the classification model are as follows:

step a, inputting the Raman spectrum data with the obvious spectral peak information into a convolutional layer and a max-pooling layer for spectrum data dimension reduction;

step b, inputting the spectrum after dimension reduction into two convolutional layers, and extracting multi-scale local spectrum feature data X_{c1};

step c, inputting the extracted multi-scale local spectrum feature data into the two convolutional layer and a multi-head self-attention layer, and performing information fusion of correlation between a local spectrum feature and a global spectrum feature;

step d, inputting the feature after information fusion into an adaptive average pooling layer, and performing feature dimension reduction; and inputting the feature after dimension reduction into a fully-connected layer, so as to obtain a final category probability output;

and step e, adjusting network model parameters according to a loss function to obtain the classification model of final network global optimal solution parameters.

In step a, first, the Raman spectrum data with the obvious spectral peak information is inputted into a convolutional layer to obtain first feature data X_{c} of which the spectrum dimensions are reduced by half; then, the first feature data is inputted into a max-pooling layer, and a max-pooling operation is performed to obtain second feature data Xₚ of which the spectrum dimensions are further reduced by half.

The specific process of step c is as follows:

step c_1, constituting a feature fusion module by two convolutional layers which are in residual connection and a multi-head self-attention layer;

step c_2, successively taking the output of a previous feature fusion module as the input of a next feature fusion module, and constructing three multi-scale feature fusion modules, wherein the number of one-dimensional convolution kernels used by the two convolutional layers in each module is doubled, and in each module, the size of feature data segmentation and the number of attention heads of the multi-head self-attention layer are set to be different;

step c_3, inputting the multi-scale local spectrum feature data X_{c1} into a convolutional layer in a single module to obtain multi-scale feature data X_{c2} of which the number of the spectrum dimensions are reduced by half and the number of multi-scale features are doubled;

and step c-4, inputting the multi-scale feature data X_{c2} into a multi-head self-attention layer in a single module to obtain multi-scale feature fusion data X_{F}.

The specific formula for acquiring the multi-scale feature fusion data X_{F} is as follows:

$$\begin{matrix}{X}_{F} = MSA ( z ) = [ {SA}_{1} ( z ) ; {SA}_{2} ( z ) ; … ; {SA}_{h} ( z ) ] \\ SA ( Q , K , V ) = Soft max ( Q {K}^{T} / \sqrt{{d}_{h}} ) V \\ [ Q , K , V ] = z {W}_{Q K V} \\ z = [ {X}_{c 2 ; p}^{1} ; {X}_{c 2 ; p}^{2} ; … ; {X}_{c 2 ; p}^{n} ] + {E}_{p o s}\end{matrix}$$

wherein, X_{c2;p}ⁿ ∈ R^{p×d}, Eₚₒₛ ∈R^{n×d}, d represents the number of one-dimensional convolution kernels in a convolutional layer, X_{c2;p}ⁿ represents dividing the multi-scale feature data X_{c2} into nth small feature blocks with the size of p, and Eₚₒₛ represents position information of each small feature block; z ∈ R^{n×d}, W_{OKV} ∈ R^{d×3d}^{ₕ}, h represents the, number of the multi-head self-attention mechanism heads, dₕ represents the number of one-dimensional convolution kernels contained in each multi-head self-attention mechanism head, z represents encoding information after all the pieces of multi-scale feature data X_{c2} are divided into blocks, W_{QKV} represents z initialized weight information, and QKV of each small feature block is obtained after a vector dot multiplication operation is performed on the two; SA represents a self-attention weight of each small feature block, and MSA represents a final multi-head self-attention weight of each small feature block.

The specific process of step d is as follows:

inputting the multi-scale feature fusion data X_{F} into an adaptive average pooling layer to obtain feature data Xₐ with the size of a set number of feature dimensions, and inputting the feature data Xₐ into a fully-connected layer to obtain a final category probability output, wherein the number of output neurons of the fully-connected layer is set as the number of categories of a target classification task.

A Raman spectrum-based species blood and semen classification method, comprising the following steps:

step 1, acquiring blood and semen Raman spectrum data samples of several species as a training set and a test set;

step 2, applying the aforementioned Raman spectrum classification method, training a classification model on the basis of the data acquired in step 1, and verifying and optimizing the classification model by applying the test set;

and step 3, inputting the real-time acquired blood and semen Raman spectrum data of the various species into the optimized classification model so as to acquire a final classification result.

A Raman spectrum-based species classification method, comprising the following steps:

step 1, acquiring Raman spectrum data samples of arbitrary attributes of several species as a training set and a test set;

step 2, applying the aforementioned Raman spectrum classification method, training a species classification model on the basis of the data acquired in step 1, and verifying and optimizing the species classification model by applying a test set;

and step 3, inputting the real-time acquired Raman spectrum data of the various species into the optimized species classification model so as to acquire a final species classification result.

A computer storage medium, wherein the computer storage medium stores computer instructions which, when being invoked, are used for executing all or some of the steps of the aforementioned method.

compared with the prior art, the present invention has the following beneficial effects:

1. a convolutional neural network and multi-head self-attention mechanism combined Raman spectrum classification method, in which the spectrum can be subjected to convolution calculation to obtain local peak feature information, and can be subjected to multi-head self-attention calculation to obtain global peak correlation information, and the spectrum has the effect of multi-scale feature fusion. Compared with the existing Raman spectrum classification method, effective combination of the two network structures can achieve the effect of complementary advantages, so that the local feature peak and global peak correlation of the Raman spectrum can be effectively combined, so as to achieve a more accurate classification performance, thereby improving the classification accuracy.

2. Accurate and rapid prediction of the rare species corresponding to blood and semen is achieved, thereby reducing the crime phenomenon of illegal smuggling of rare animals and effectively protecting the national biological resources. Compared with a traditional machine learning method, a better classification performance is obtained in a large-scale Raman spectrum data set. In a reflective Raman spectrum data set of blood and semen of 40 species, the classification accuracy rate may reach 99.2%, providing a new method for the classification detection of the species blood and semen.

3. The method can not only be used for the classification of the species blood and semen, but also be used in other biological classification and identification fields, achieving a high practical value.

### BRIEF DESCRIPTION OF THE DRAWINGS

FIG. 1 is a flowchart of network computing of the method and a corresponding actual network structure according to the present invention.

FIG. 2 shows the original spectrogram of the blood reflective Raman spectrum of a single species, i.e. horse, according to the present invention.

FIG. 3 shows the spectrogram after preprocessing of the blood reflective Raman spectrum of a single species, i.e. horse, according to the present invention.

FIG. 4 is a graph of a network training loss and an accuracy rate according to the present invention.

FIG. 5 is a result diagram of a blood and semen confusion matrix of using the network in a test set to predict an unknown species after the completion of the training according to the present invention.

### DETAILED DESCRIPTION

The structure and working process of the present invention will be further described in combination with the accompanying drawings.

The present invention provides a convolutional neural network and multi-head self-attention mechanism combined Raman spectrum classification method, in which the spectrum can be subjected to convolution calculation to obtain local peak feature information, and can be subjected to multi-head self-attention calculation to obtain global peak correlation information, and the spectrum has the effect of multi-scale feature fusion, effective combination of the two network structures can achieve the effect of complementary advantages, thereby improving the classification. By means of the method, blood and semen of various species can be accurately classified simply and quickly, thereby reducing the crime phenomenon of illegal smuggling of rare animals and effectively protecting the national biological resources. In addition, the method can not only be applied to the classification of the species blood and semen, but also can be applied to other biological classification and identification fields.

A Raman spectrum classification method, first, acquiring several pieces of Raman spectrum data as a training set and a test set; second, after performing a series of preprocessing on the Raman spectrum data according to the existing spectrum quality, acquiring Raman spectrum data with obvious spectral peak information;

then, inputting the Raman spectrum data with the obvious spectral peak information into a one-dimensional convolution and multi-head self-attention mechanism combined neural network model to train a classification model;

and finally, inputting the Raman spectrum test set data to the trained classification model to obtain a final classification result.

A Raman spectrum-based species blood and semen classification method, comprising the following steps:

step 1, acquiring blood and semen Raman spectrum data samples of several species as a training set and a test set;

step 2, applying the aforementioned Raman spectrum classification method, training a classification model on the basis of the data acquired in step 1, and verifying and optimizing the classification model by applying the test set;

and step 3, inputting the real-time acquired blood and semen Raman spectrum data of the various species into the optimized classification model so as to acquire a final classification result.

A Raman spectrum-based species classification method, comprising the following steps:

step 1, acquiring Raman spectrum data samples of arbitrary attributes of several species as a training set and a test set;

step 2, applying the aforementioned Raman spectrum classification method, training a species classification model on the basis of the data acquired in step 1, and verifying and optimizing the species classification model by applying a test set;

and step 3, inputting the real-time acquired Raman spectrum data of the various species into the optimized species classification model so as to acquire a final species classification result.

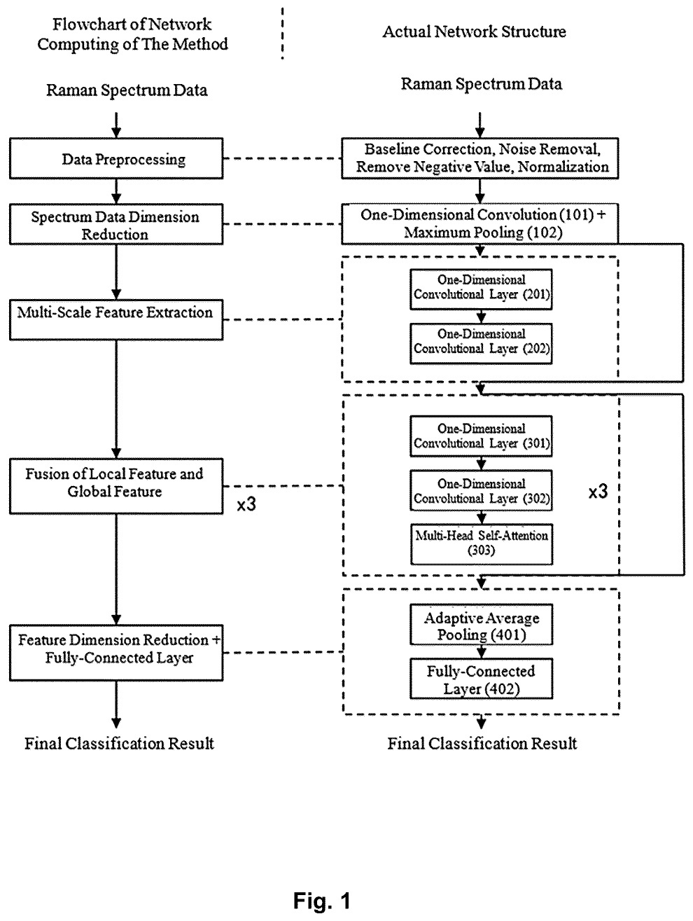

Specific embodiments, as shown in FIG. 1 to FIG. 5,

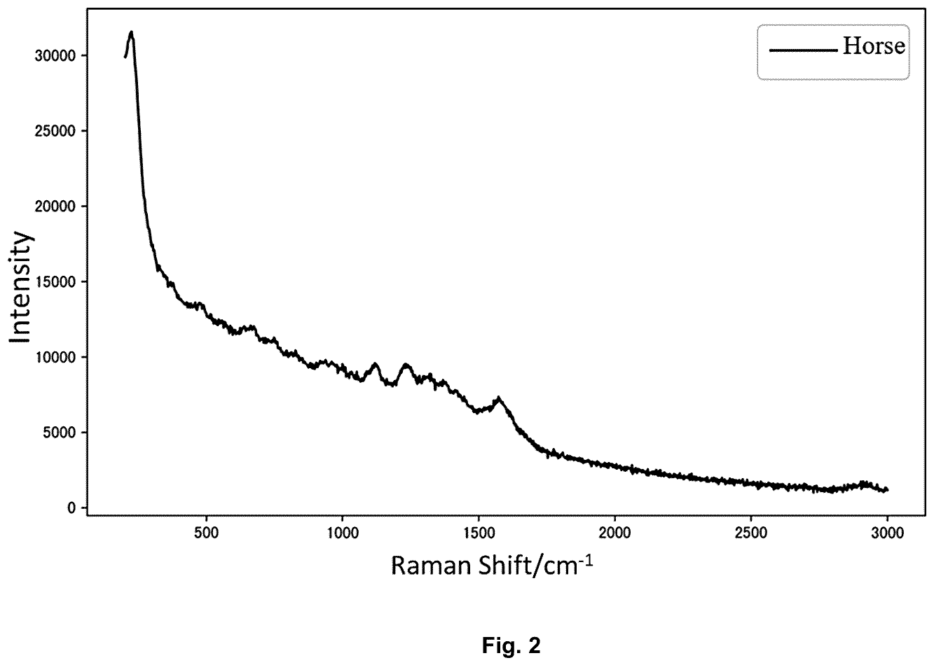

The data set used in the present embodiment is from research on special biological resources monitoring and tracing technology in national research and development plans; the blood reflective Raman spectrum of a single species, i.e. horse, is visualized as shown in FIG. 2; the data comprises 40 species of national rare animals such as kangaroos, Japanese crane, golden monkey, and horse, and common animals in daily life; and there are 5229 pieces of reflective Raman spectrum data in total, in which 9.8% (515 pieces) of the data is taken as a test set.

As shown in FIG. 1, the method includes the following steps:

steps S1, performing preprocessing on spectrum data according to the existing spectrum quality, the specific implementation steps being as follows:

step S11, determining whether to perform baseline correction processing on a Raman spectrum according to there is a baseline drift phenomenon in Raman spectrum data, in which in this embodiment, there is a baseline drift phenomenon in the Raman spectrum, and a baseline correction processing needs to be performed;

step S12, according to whether there is a phenomenon that an instrument noise exists in a Raman spectrum acquisition process, such that spectrum lines fluctuate sharply and the spectral peak is not obvious, determining whether to perform noise removal processing on the Raman spectrum, in which in this embodiment, there is a noise in the Raman spectrum, and it is necessary to perform a noise removal processing;

step S13, discarding spectrum dots of which the spectrum intensity is a negative value, and replacing same with 0, in which in this embodiment, a small number of spectrum dots have a negative value phenomenon, and are replaced with 0;

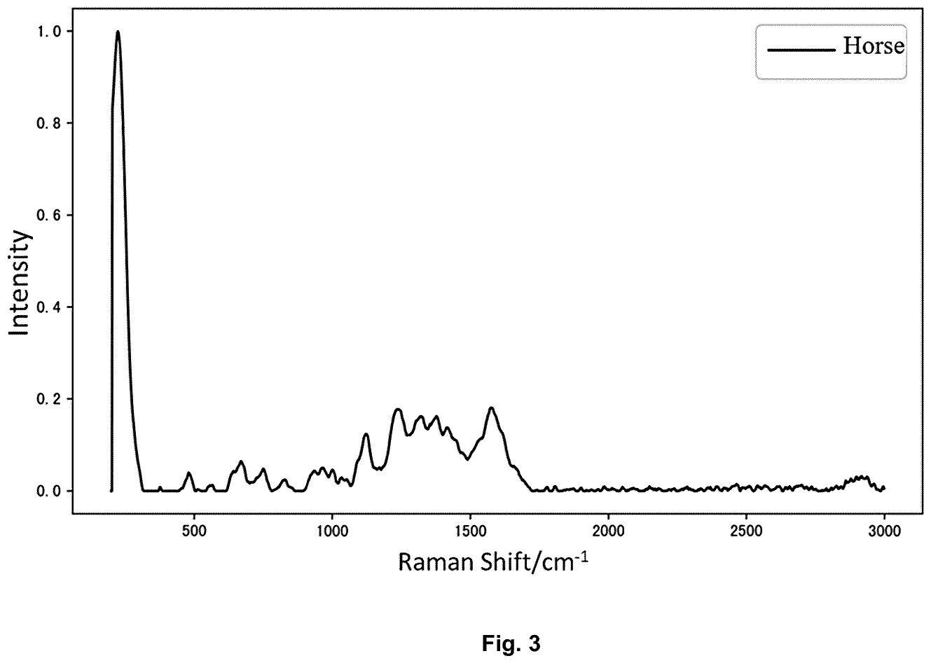

step S14, normalizing the Raman spectrum, so as to accelerate the convergence speed of the network training, in which in this example, Raman spectrum intensity is normalized to [0,1], the Raman spectrum dimension is (1, 1400), and the blood spectrum after preprocessing of the single species, i.e. horse, is shown in FIG. 3;

step S2, inputting the spectrum data into a convolutional layer and a max-pooling layer for spectrum data dimension reduction, the specific implementation steps being as follows:

step S21, preprocessing the spectrum to obtain spectrum data, and inputting same into a convolutional layer (101), in which the convolutional layer (101) uses 16 kinds of one-dimensional convolution kernels with a feature size of 7*1, the convolution step length is 2, and spectrum filling is 6; perform a convolution operation on the input data, so as to obtain feature data X_{c} with the 16 spectrum dimensions reduced by half; and in this embodiment, the preprocessed spectrum data X_{c} is inputted into the convolutional layer (101) to obtain feature data X_{c} of the dimension (16, 700);

step S22, inputting the feature data X_{c} into the max-pooling layer (102), in which the max-pooling layer (102) uses a one-dimensional filter with a feature size of 3*1, the pooling stride is 2, and spectrum filling is 2; and performing a max-pooling operation on the input data, so as to obtain feature data Xₚ with the 16 spectrum dimensions further reduced by half, in which in this embodiment, feature data X_{c} is inputted into the max-pooling layer (102) to obtain feature data Xₚ with the dimension (16, 350);

step S3, inputting the spectrum after dimension reduction into two convolutional layers, and extracting multi-scale local spectrum feature data, the specific implementation steps being as follows:

step S31: inputting the spectrum data Xₚ after dimension reduction into two convolutional layers (201, 202) which are in residual connection, in which the two convolutional layers (201, 202) both use 32 kinds of one-dimensional convolution kernels with a feature size of 3*1, the convolution stride is 1, and spectrum filling is 2; and performing convolution operation on the input data, so as to obtain multi-scale feature data X_{c1} with 32 spectrum dimensions unchanged, in which in this embodiment, the feature data Xₚ after dimension reduction is inputted into the two convolutional layers (201, 202) to obtain multi-scale feature data X_{c1} with the dimension (32, 350);

step S4, inputting the extracted features into the two convolutional layers and a multi-head self-attention layer for information fusion of correlation between a local spectrum feature and a global spectrum feature, the specific implementation steps being as follows:

step S41, constituting a multi-scale feature fusion module by the two convolutional layers (301, 302) which are in residual connection and a multi-head self-attention layer (303), taking the output of a previous module as the input of a next module, and the step is repeated three times. In this embodiment, three multi-scale feature fusion modules are constructed, and the multi-scale feature data X_{c1} is inputted into the three multi-scale feature fusion modules, the specific steps being as follows:

step S411, constructing three multi-scale feature fusion modules, in which the number of one-dimensional convolution kernels used by the two convolutional layers (301, 302) in each module is doubled, and inputting the multi-scale feature data X_{c1} into a single module to obtain feature data X₂ of which the number of the spectrum dimensions are reduced by half and the number of multi-scale features are doubled. In this embodiment, the three multi-scale feature fusion modules are constructed, in which the two convolutional layers (301, 302) respectively use 64, 128, 256 kinds of one-dimensional convolution kernels with a feature size of 3*1, the convolution strides of each two convolutional layers (301, 302) are both 2, 1, and spectrum filling is both 2.

Step S412, constructing three multi-scale feature fusion modules, in which in each module, the size of feature data segmentation and the number of attention heads of the multi-head self-attention (MSA) layer 303 are set to be different, and inputting the multi-scale feature data X_{c2} into the multi-head self-attention layer in a single module to obtain multi-scale feature fusion data X_{F}, in which

$$\begin{matrix}{X}_{F} = MSA ( z ) = [ {SA}_{1} ( z ) ; {SA}_{2} ( z ) ; … ; {SA}_{h} ( z ) ] \\ SA ( Q , K , V ) = Soft max ( Q {K}^{T} / \sqrt{{d}_{h}} ) V \\ [ Q , K , V ] = z {W}_{Q K V} \\ z = [ {X}_{c 2 ; p}^{1} ; {X}_{c 2 ; p}^{2} ; … ; {X}_{c 2 ; p}^{n} ] + {E}_{p o s}\end{matrix}$$

in which X_{c2:p}ⁿ ∈ R^{p×d}, Eₚₒₛ 531 R^{n×d}, d represents the number of one-dimensional convolution kernels in a convolutional layer, X_{c2:p}ⁿ represents dividing the multi-scale feature data X_{c2} into n th small feature blocks with the size of p, and Eₚₒₛ represents position information of each small feature block; z ∈ R^{n×d}, W_{QKV} ∈ R^{d×3d}^{ₕ}, h represents the number of the multi-head self-attention mechanism heads, dₕ represents the number of one-dimensional convolution kernels contained in each multi-head self-attention mechanism head, z represents encoding information after all the pieces of multi-scale feature data X_{c2} are divided into blocks, W_{QKV} represents z initialized weight information, and QKV of each small feature block is obtained after a vector dot multiplication operation is performed on the two; SA represents a self-attention weight of each small feature block, and MSA represents a final multi-head self-attention weight of each small feature block. In this embodiment, the multi-head self-attention layer (303) divides the feature data into small feature blocks with the size of 35, 44, and 22, and the number of attention heads is set to be 1, 2, and 4, respectively. Finally, the multi-scale feature data X_{c1} is inputted into the three modules to obtain multi-scale feature fusion data X_{F} with the feature dimension (64, 175), (128, 88) and (256, 44), respectively.

Step S5, inputting the feature after information fusion into an adaptive average pooling layer for feature dimension reduction; and inputting the feature after dimension reduction into a fully-connected layer, so as to obtain a final category probability output. The specific steps are as follows:

step S51: inputting the multi-scale feature fusion data X_{F} into an adaptive average pooling layer (401), in which the output size of the adaptive average pooling is set to be 7 to perform adaptive average pooling calculation on the input feature data X_{F} so as to obtain feature data Xₐ with a feature dimension size of 7. In this embodiment, the multi-scale feature fusion data X_{F} with the final data dimension (256, 44) is inputted into an adaptive average pooling layer to obtain feature data Xₐ with the data dimension (256, 7);

step S52, inputting the feature data Xₐ into a fully-connected layer (402), in which the number of output neurons of the fully-connected layer is set as the number of categories of a target classification task, so as to obtain a final category probability output. In this embodiment, the number of categories of a target classification task is 40, and the feature data Xₐ is inputted into a fully-connected layer to obtain 40 categories of probability outputs, in which the maximum probability value is a model prediction classification.

Step S6, in a network training process, according to a loss function, adjusting a network model parameter by means of an AdamW optimization algorithm, so as to obtain final network global optimal solution parameters. The specific steps are as follows:

step S61, selecting cross entropy loss functions as the network training loss function for evaluating a gap between a model prediction result and a true result. In this embodiment, 40 categories of cross entropy loss functions are used.

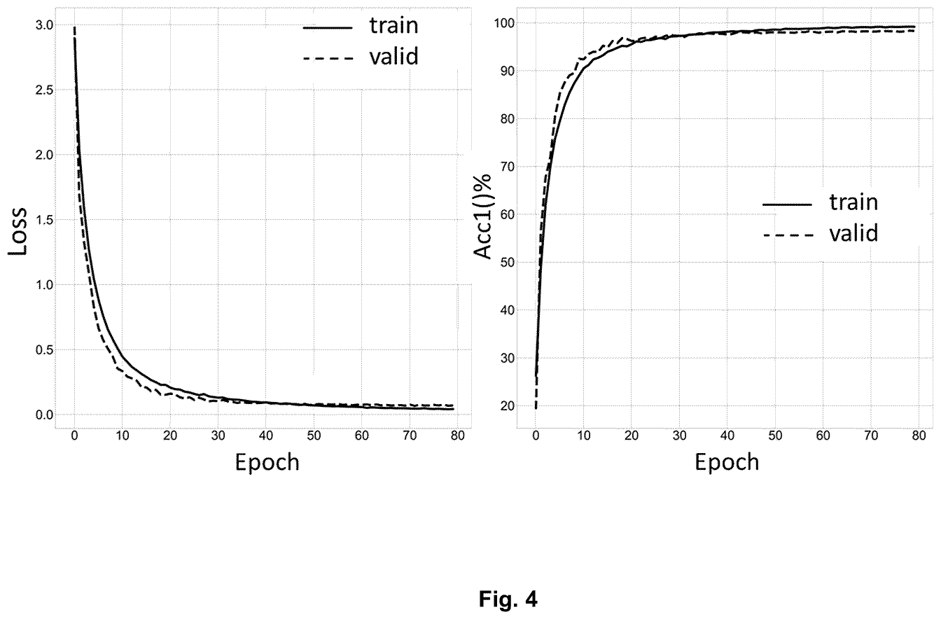

step S62, calculating and obtaining a derivative of a category distance according to the loss functions, and continuously iteratively updating the network training parameters by using the AdamW optimization algorithm, so as to obtain a fitted global optimal parameter result. In this embodiment, the network training 80 epochs achieves the best convergence effect with the smallest model loss value. The specific training loss and accuracy rate curves are shown in FIG. 4.

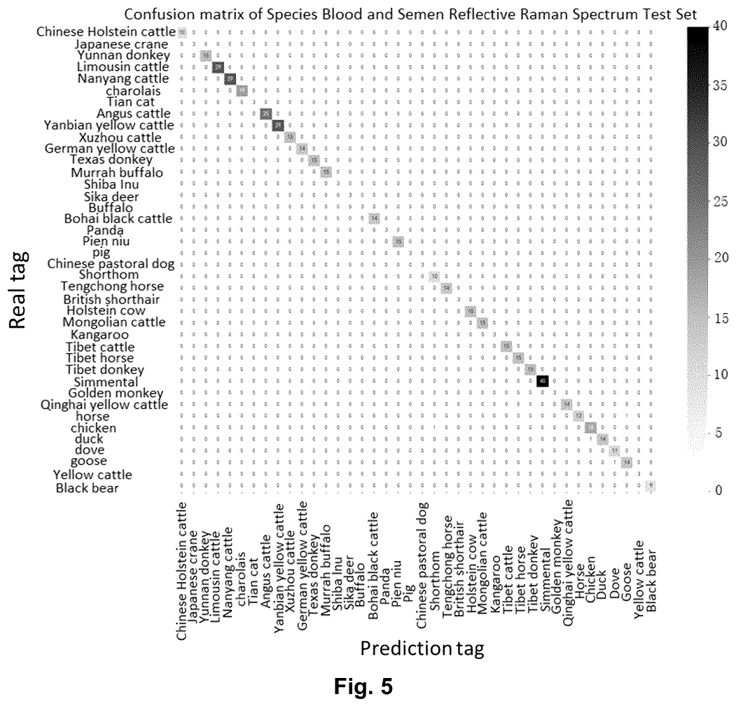

Step S7, after the training of the network model is completed, using same in an actual spectrum detection and classification task. In this embodiment, test set data is inputted into a trained network model, and a classification result is outputted. A total of 515 pieces of sample data are contained in the test set, and results of various prediction confusion matrices of the model are shown in FIG. 5.

In the present invention, on the basis of the Raman spectrum data, Raman spectrum classification is completed by means of steps such as data preprocessing, spectrum data dimension reduction, multi-scale local feature extraction, multi-scale information fusion and fully-connected layer mapping, so that blood and semen of various species can be accurately classified simply and quickly, thereby reducing the crime phenomenon of illegal smuggling of rare animals and effectively protecting the national biological resources.

The person skilled in the art should understand that the person skilled in the art can realize variations in combination with the prior art as well as the above embodiments, and such variations do not affect the substance of the present scheme, and will not be repeated herein.

It is to be understood that the present solution is not limited to the above specific embodiments, in which the equipment and structure not described in detail should be understood as implemented in the ordinary way in the field; any skilled person familiar with the field, without departing from the scope of the present solution, can make many possible changes and modifications to the technical solution of the present solution by utilizing the above disclosed methods and technical contents, or modifying them into equivalent embodiments with equivalent changes, which does not affect the substance of the present scheme. Therefore, any simple modifications, equivalent changes and modifications to the above embodiments based on the technical substance of the present solution, without departing from the content of the technical solution of the present solution, still fall within the scope of protection of the technical solution of the present solution.

What is claimed is:

1. A Raman spectrum classification method, comprising:
first, acquiring several pieces of Raman spectrum data as a training set and a test set;
second, after performing a series of preprocessing on the Raman spectrum data according to the existing spectrum quality, acquiring Raman spectrum data with obvious spectral peak information;
then, inputting the Raman spectrum data with the obvious spectral peak information into a one-dimensional convolution and multi-head self-attention mechanism combined neural network model to train a classification model;
and finally, inputting the Raman spectrum test set data to the trained classification model to obtain a final classification result.

2. The Raman spectrum classification method according to claim 1, wherein the specific steps of performing preprocessing the spectrum data are as follows:
step 1, determining whether there is a baseline drift phenomenon in the Raman spectrum data, and if there is a baseline drift phenomenon, performing baseline correction processing;
step 2, determining whether there is an instrument noise in the Raman spectrum data acquisition process, and if there is a noise, performing noise removal processing on the Raman spectrum data;
step 3, replacing a spectrum dot with a spectrum intensity of a negative value with 0;
and step 4, normalizing the Raman spectrum obtained in step 3, and acquiring the Raman spectrum data X with obvious spectral peak information.

3. The Raman spectrum classification method according to claim 2, wherein the specific steps of training the classification model are as follows:
step a, inputting the Raman spectrum data with the obvious spectral peak information into a convolutional layer and a max-pooling layer for spectrum data dimension reduction;
step b, inputting the spectrum after dimension reduction into two convolutional layers, and extracting multi-scale local spectrum feature data X_{c1};
step c, inputting the extracted multi-scale local spectrum feature data into the two convolutional layer and a multi-head self-attention layer, and performing information fusion of correlation between a local spectrum feature and a global spectrum feature;
step d, inputting the feature after information fusion into an adaptive average pooling layer, and performing feature dimension reduction; and inputting the feature after dimension reduction into a fully-connected layer, so as to obtain a final category probability output;
and step e, adjusting network model parameters according to a loss function to obtain the classification model of final network global optimal solution parameters.

4. The Raman spectrum classification method according to claim 3, wherein: in step a, first, the Raman spectrum data with the obvious spectral peak information is inputted into a convolutional layer to obtain first feature data X_{c} of which the spectrum dimensions are reduced by half; then, the first feature data is inputted into a max-pooling layer, and a max-pooling operation is performed to obtain second feature data Xₚ of which the spectrum dimensions are further reduced by half.

5. The Raman spectrum classification method according to claim 4, wherein the specific process of step c is as follows:
step c_1, constituting a feature fusion module by two convolutional layers which are in residual connection and a multi-head self-attention layer;
step c_2, successively taking the output of a previous feature fusion module as the input of a next feature fusion module, and constructing three multi-scale feature fusion modules, wherein the number of one-dimensional convolution kernels used by the two convolutional layers in each module is doubled, and in each module, the size of feature data segmentation and the number of attention heads of the multi-head self-attention layer are set to be different;
step c_3, inputting the multi-scale local spectrum feature data X_{c1} into a convolutional layer in a single module to obtain multi-scale feature data X_{c2} of which the number of the spectrum dimensions are reduced by half and the number of multi-scale features are doubled;
and step c-4, inputting the multi-scale feature data X_{c2} into a multi-head self-attention layer in a single module to obtain multi-scale feature fusion data X_{F}.

6. The Raman spectrum classification method according to claim 5, wherein: the specific formula for acquiring the multi-scale feature fusion data X_{F} is as follows:
$$\begin{matrix}{X}_{F} = MSA ( z ) = [ {SA}_{1} ( z ) ; {SA}_{2} ( z ) ; … ; {SA}_{h} ( z ) ] \\ SA ( Q , K , V ) = Soft max ( Q {K}^{T} / \sqrt{{d}_{h}} ) V \\ [ Q , K , V ] = z {W}_{Q K V} \\ z = [ {X}_{c 2 ; p}^{1} ; {X}_{c 2 ; p}^{2} ; … ; {X}_{c 2 ; p}^{n} ] + {E}_{p o s}\end{matrix}$$
wherein, X_{c2;p}ⁿ ∈ R^{p×d}, Eₚₒₛ ∈ R^{n×d}, d represents the number of one-dimensional, convolution kernels in a convolutional layer, X_{c2;p}ⁿ represents dividing the multi-scale feature data X_{c2} into nth small feature blocks with the size of p, and Eₚₒₛ represents position information of each small feature block; z ∈ R^{n×d}, W_{QKV} ∈ R^{d×3d}^{ₕ}, h represents the number of the multi-head self-attention mechanism heads, dₕ represents the number of one-dimensional convolution kernels contained in each multi-head self-attention mechanism head, z represents encoding information after all the pieces of multi-scale feature data X_{c2} are divided into blocks, W_{QKV} represents z initialized weight information, and QKV of each small feature block is obtained after a vector dot multiplication operation is performed on the two; SA represents a self-attention weight of each small feature block, and MSA represents a final multi-head self-attention weight of each small feature block.

7. The Raman spectrum classification method according to claim 3, wherein: the specific process of step d is as follows:
inputting the multi-scale feature fusion data X_{F} into an adaptive average pooling layer to obtain feature data Xₐ with the size of a set number of feature dimensions, and inputting the feature data Xₐ into a fully-connected layer to obtain a final category probability output, wherein the number of output neurons of the fully-connected layer is set as the number of categories of a target classification task.

8. A Raman spectrum-based species blood and semen classification method, comprising the following steps:
step 1, acquiring blood and semen Raman spectrum data samples of several species as a training set and a test set;
step 2, applying the Raman spectrum classification method of claim 1, training a classification model on the basis of the data acquired in step 1, and verifying and optimizing the classification model by applying the test set;
and step 3, inputting the real-time acquired blood and semen Raman spectrum data of the various species into the optimized classification model so as to acquire a final classification result.

9. A Raman spectrum-based species classification method, comprising the following steps:
step 1, acquiring Raman spectrum data samples of arbitrary attributes of several species as a training set and a test set;
step 2, applying the Raman spectrum classification method of claim 1, training a species classification model on the basis of the data acquired in step 1, and verifying and optimizing the species classification model by applying a test set;
and step 3, inputting the real-time acquired Raman spectrum data of the various species into the optimized species classification model so as to acquire a final species classification result.

10. A computer storage medium, wherein the computer storage medium stores computer instructions which, when being invoked, are used for executing all or some of the steps of the method of claim 1.
